(19) United States
(12) Patent Application Publication
(10) Pub. No.: US 2026/0137505 A1
(43) Pub. Date: May 21, 2026

(54) CYCLIC LENS SURFACE WITH EXTENDED DEPTH OF FOCUS

(71) Applicant: EyeQ Technologies, Inc., Reno, NV (US)

(72) Inventor: Matthew Schuster, Portland, OR (US)

(21) Appl. No.: 18/994,992

(22) PCT Filed: Jul. 18, 2023
(86) PCT No.: PCT/US2023/027985

§ 371 Date: Jan. 15, 2025

Related U.S. Application Data
(60) Provisional application No. 63/390,213, filed on Jul. 18, 2022.
(60) Provisional application No. 63/402,809, filed on Aug. 31, 2022.

(51) Int. Cl. A61F 2/16

(52) U.S. Cl. CPC A61F 2/1618; A61F 2/164

(57) ABSTRACT

A lens body, such as an intraocular lens, is configured to be implanted in an eye. An outer surface of the lens includes a combined cyclic surface, wherein the combined cyclic surface has a first portion periodically optimized at first focal length, a second portion periodically optimized at a second focal length, and a third portion that periodically connects the first and second portions. The third portion can be defined by a periodic function that varies in amplitude and frequency.

### CROSS-REFERENCE TO RELATED APPLICATIONS

This application claims priority to U.S. Patent Application Nos. 63/390,213, filed on Jul. 18, 2022, and 63/402,809, filed on Aug. 31, 2022, entitled “CYCLIC LENS SURFACE WITH EXTENDED DEPTH OF FOCUS”, the contents of which are hereby incorporated by reference herein in their entirety.

### BACKGROUND

Conventional optical lens solutions for extending depth of focus while eliminating common drawbacks include diffractive and refractive multifocal lenses having discrete zones. Other conventional solutions include aspheric lenses with a power profile that varies over the radius of the lens and small aperture (e.g., pinhole) lenses. In the case of the aspheric lens shape, the degree to which the power varies over the radius is set by varying a conic constant K and the nth order aspheric coefficient terms.

Such techniques have drawbacks. For example, in the case of an intraocular lens (IOL), the placement of discrete zones is sensitive to pupil size. If a particular power zone is either exposed by a dilated pupil or covered by a constricted pupil, it will dramatically change the resultant power of the IOL. In another example, multi-zone configurations by definition introduce unwanted visual artifacts such as halos. For an object at a given distance, one zone might be in excellent or good focus while the other zones may be at somewhat less than perfect focus. This introduces multiple images and halos around the primary object. Further, with a small number of zones, the halos become more pronounced and distinct. Further, restricted aperture IOLs have suboptimal low contrast resolution due to limited light gathering power and relatively dim images projected onto the retina.

There is a need for lens configurations that alleviate or eliminate the aforementioned drawbacks.

### SUMMARY

Disclosed is a lens, such as an intraocular lens, configured for a continuously varied focal length. The lens has a shape with an outer anterior and/or posterior surface defined by a combination of at least one baseline lens shape wherein the shape is combined with a shape defined by a periodic function wherein the periodic function can be modulated in amplitude and period. This results in a lens having a shape with continuously variable power moving along a surface of the lens such as radially outward relative to an optic axis. The baseline lens shape can vary and can be for example spherical, aspheric, or any other appropriate lens definition. In another embodiment, the lens is not defined by a baseline shape but is rather initially defined by a one or more slope curves (i.e., slope of curvature of an outer surface of the lens) that define desired or optimal power levels of the lens such as in a bespoke manner. The slope curve(s) can be connected by one or more additional slope curves defined by a periodic function, a piecewise function, and arbitrary function or any function with transitions in slope curve from one location to another being arbitrary or predefined. The slope curves are then mathematically integrated to define a curvature of an outer surface of the lens.

In the case of an IOL, in an non-limiting embodiment, the lens is shaped to provide the lens with an optimal lens power for a first vision (e.g., distance vision) and a second lens power for a second vision (e.g., near vision.) The lens can further be configured with powers for multiple visions lengths beyond distance vision and near vision. A radius of curvature of an outer lens surface shape for the two optimal lens powers is necessarily different with the distance vision lens having a larger radius of curvature than a radius of curvature of the near vision lens. By extension, the slope (such as a first derivative) of the curve of the distance vision lens increases more gradually moving away from the center optic axis than the slope of the curve of the near vision lens. In one aspect of this disclosure, a periodic surface wave that defines a variation of the slope/radius of curvature of the lens provides the lens with a curvature that repeatedly varies from distance optimized curvature to near optimized curvature over the radius of the lens. In another aspect, the variation of the slope/radius of curvature is periodic and irregular or random.

The type of lens that employs the features disclosed herein can vary. The lens can be any lens that utilizes a cyclic surface for enhanced depth of focus and reduced unwanted aberrations. In a non-limiting example, the lens comprises an IOL. In another non-limiting example, the lens comprises an endoscope with a fixed lens system.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 shows a diagram showing various radii of curvature of a lens optimized at different distances.

FIG. 2 shows a graph of the slope of curvature of a lens optimized at different distances as a function of distance r from the optic axis of the lens.

FIG. 3 shows the slope of the combined outer surface lens function S′(r) that defines the combined outer lens surface.

FIGS. 4 and 4A each show an example representation of a combined cyclic surface as defined by the combined lens function S′(r).

FIG. 4B shows a graphical representation of the surface as a function of radius.

FIG. 5 shows a graphical representation of a first line of the slope of curvature of far vision baseline outer shape and a second line of the slope of curvature of far vision baseline outer shape as function of radial distance of the lens from the optic axis.

FIG. 6 shows a period function defining the slopes that transition between the transition points of a lens outer surface.

FIG. 7A shows a histogram of value of slopes of curvature across the entire lens with the horizontal axis being slope value and the vertical axis being quantity of occurrence.

FIG. 7B shows another histogram that represents curvature of the outer surface of the lens.

FIGS. 8A-8C illustrate a non-limiting example of a lens comprising an IOL.

FIG. 9 depicts a block diagram illustrating an example of a computing system consistent with implementations of the current subject matter.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

Disclosed is a lens having an outer surface (sometimes referred to as a “combined surface, combined cyclic surface or combined outer surface”) defined by one or more baseline lens surface shapes combined with a surface shape defined by a function, such as a periodic function (such as a sinusoidal function) that can be modulated in amplitude and period. In another embodiment, the function is a chirp function, a logarithmically varying function, a piecewise function, or a random variation. As mentioned, the base shape can vary and can be for example, a spheric baseline surface, an aspheric surface, a combination thereof, or any surface described by another mathematic function. The periodic function can also vary. For example, the periodic function can be a simple modulated periodic function such a sin or cosine function. In another example, the periodic function is arbitrarily complex periodic function comprised of an arbitrarily large number of sin and cosine terms. An additional flexibility in the periodic function allows for complete control over all shape parameters of the periodicity (rise and decay steepness, dwell at extrema, variations in amplitude and periodicity, etc.). The combined outer surface can be an anterior surface of the lens, a posterior surface of the lens, a portion of the anterior surface, and/or a portion of the posterior surface. For example, the combined outer surface can be a region of an anterior surface (or posterior surface) that occupies a portion of the surface moving radially outward from an optic axis. Additionally, the lens can be fashioned with no base shape at all, where the modulation of the slope of the high and low power boundary condition curves fully define the resultant periodic shape. A base shape is not required, as the shape is defined by integrating the slope curve, and the slope curve is constructed by bouncing back forth between high and low power curves. Additionally, any number of mid-level power curves can be included.

The function can define an anterior and/or posterior combined surface of the lens. The combined surface is a surface defined by at least one base shape (such as a spherical, aspheric, or any other appropriate lens definition) and further defined by a predetermined function such that the outer surface periodically transitions between the base shape and a surface defined by the function. For example, the lens may be an anterior combined surface with a posterior spheric or aspheric surface. Or a posterior combined surface with a spheric or aspheric surface on the anterior side. In another example, the combined surface is on both the posterior and anterior side of the lens. In an example of an IOL, the IOL may include a conic lens (spheric or aspheric) in a center portion of IOL with a combined surface in a radially outer portion of the lens. Another embodiment employs a combined surface lens with diffractive or refractive zones for specific power emphasis.

In an embodiment, the lens has a cyclic combined outer surface, which can be an entirety or a portion of a posterior surface and/or an anterior surface. The cyclic outer surface has a first portion periodically defined by the base shape optimized at a first focal length, a second portion periodically defined by the base shape optimized at a second focal length, and a third portion that periodically connects the first and second portions, the third portion directly or indirectly defined by a function such as periodic function. Thus, moving radially outward from an optic axis of the lens, the lens outer surface periodically or cyclically oscillates between the first portion, the second portion and third portion with the third portion periodically connecting the first portion and second portion. The cyclic outer lens surface can be further defined by a base shape optimized at a third focal length that is between the first focal length and the second focal length. A lens body, such as an intraocular lens, can be configured to be implanted in an eye. An outer surface of the lens includes a combined cyclic surface, wherein the combined cyclic surface has at least first portion periodically optimized at first focal length, a second portion periodically optimized at a second focal length, and a third portion that periodically connects the first and second portions. The third portion can be defined by a periodic function that varies in amplitude and frequency or by any other function. Or the third portion can be arbitrary or bespoke.

In another embodiment, the lens is not defined by a baseline shape but is rather defined in a bespoke manner by providing one or more slope curves that define a slope of curvature of at least a portion of the lens surface. The slopes of curvature can be based at least on part on a desired power profile of the lens at one or more locations of the lens. For example, a lens designer may desire a lens that has a desired power profile at one or more locations with a different power profile at one or more other locations. The designer may specify such power profile(s) based on a slope values or slope curves for such power profiles and then connect such slope values/curves with an additional slope curve that is predefined (such as by a periodic function, arbitrary function, piecewise function, or randomly defined connection). This can result in a predefined or arbitrary number of slope definitions for the lens. The resultant, combined slope curve is then integrated to provide a curvature of at least a portion of an outer surface of the lens.

An IOL having the features disclosed herein is configured to vary power continuously rather than discretely moving along the surface of the lens, such as moving radially outward from an optic axis of the lens or in any other manner. This results in fewer caustic formations on an image projected on the retina and generally blends the power profile more effectively. The lens also allows for an arbitrary number of cycles only constrained by manufacturing limitations. This flexibility allows for extremely smooth lens power distribution.

In an embodiment, the lens includes on its anterior surface and/or posterior surface a plurality of periodic structures such as structural waves wherein an outer surface of each wave contains or defines all of the distance powers of the lens. That is, each periodic structure contains all of the lens powers in the system from high lens power to low lens power. Light transmitted through a small portion of the lens is still subject to the full range of powers. The lens further allows for an arbitrary power bandwidth. By changing the amplitude of the function (such as the periodic function), the lowest and highest power in the system can be manipulated. The lens also enables a reduction in production of halos. The lens includes numerous periodic structures each containing all possible powers in the system. This greatly enhances overall blending to dramatically reduce or eliminate halos.

In the context of an IOL, when the lens is implanted in the eye, changes in pupil diameter do not cause significant changes in performance of the lens. In a typical zone based multifocal lens, obscuration by the iris takes that obscuration portion of the system out of the optical path. This results in certain magnifications (i.e., powers) being disabled or over-emphasized as the iris constricts or dilates. This is eliminated in a lens having the disclosed features as each periodic structure of the lens contains all of the powers of the lens thus the performance will remain uniform regardless of iris dilation.

The lens can be made using traditional manufacturing techniques such a lathe with diamond cutting tools or injection molding. This technique is different from a meta-lens technique or other technologies requiring nano-scale (e.g., 1-100 nm) features. Such technologies require more exotic manufacturing techniques.

As mentioned, the disclosed lens has an outer surface (which can be the anterior and/or posterior surface of the lens and is referred to as an combined outer surface) that is defined by at least one base shape (which can be for example spherical, aspheric, or any other appropriate lens definition) combined with a shape defined by a function (such as a periodic function) modulated in amplitude and period. The resultant shape achieves a continuously variable power for the lens. The following discussion provides a description of relevant mathematics and geometry.

A mathematical description of at least one base shape of the outer surface of the lens can be described where:
R=Baseline radius of curvature of the outer surface of the lens
R=radial distance from the optic axis of the lens; and
G(r)=a function defining a lens surface pursuant to the base shape. This function can vary and can be for example spherical, aspheric could be any appropriate surface function (polynomial, etc.).

Example spherical lens profiles (or base shapes) as a function of r for a spherical lens profile and an aspheric lens profile are shown below:

Example Spherical lens profile as a function of r

$$G ( r ) = \sqrt{{R}^{2} - {r}^{2}}$$

Example Aspheric lens profile as a function of r

$$G ( r ) = \frac{{r}^{2}}{R ( 1 + \sqrt{1 - ( 1 + k ) \frac{{r}^{2}}{{R}^{2}}} )} + {A}_{4} {r}^{4} + {A}_{6} {r}^{6} … + {A}_{n} {r}^{n}$$

A first derivate (G′(r)) of the function defining the lens surface is obtained.

G′(r)=is the first derivative of the function defining the lens surface. Put differently, G(r) is the slope of the lens outer surface at a given location as a function of r.

H(r)=a function (such as a periodic function) defining an outer lens surface and allowing control over amplitude and period including the relative slope of each period in the function. By the Fourier theorem, any periodic function that is reasonably continuous can be expressed as a sum of a series of sine and cosine terms. The implication is that any periodic function can be represented by a combination of sine and cosine terms. As described below, the function can further be a chirp function, a non-linear chirp function (such as a logarithmically varying function), a piecewise function, or a randomly varying function.

A representative example is shown below where:
Amplitude constant=a term to vary the amplitude of the periodic function (in this case the amplitude changes proportionate to the distance r from the optic axis of the lens); and
Frequency constant=a term which varies the period of the function. A larger frequency constant increases the frequency of the periodic function and results in more periods combined with the underlying base shape profile described above.

$$H ( r ) := \frac{r}{Amplitude constant} Cos [ Frequency Constant \times r ]$$

Where
H′(r)=The first derivative of an appropriate periodic function as described above. Put differently, H′(r) is the slope of the above periodic function that defines a portion of the lens surface; and
(r)=G(r)+H(r) is a function combing the base shape of the lens surface defined above with the function defined above to achieve the combined outer surface shape. The combination G(r)+H(r) could be a sum or convolution or any combination of the above functions that combines the periodicity and amplitude of H(r) with the underlying (baseline) lens shape of G(r). The effect is to combine the periodic surface with the underlying lens surface (baseline shape). An example function S (r) is shown below:

$$S ( r ) = \frac{r}{Amplitude constant} Cos [ Frequency Constant \times r ] + \sqrt{{R}^{2} - {r}^{2}}$$

S′(r)=First derivative of the function combining the lens surface function G(r) with the periodic function H(r) wherein S′(r) defines a slope of the outer combined lens surface a function of r (the radial distance from the optic axis of the lens.)

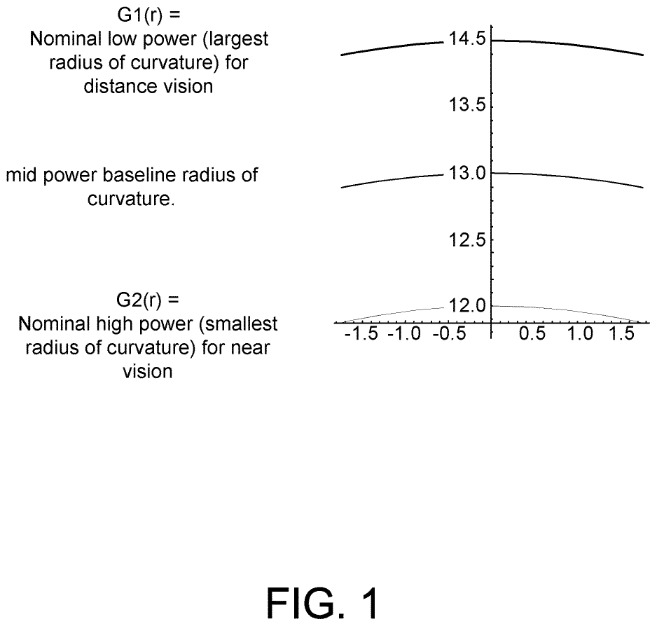

FIG. 1 shows a diagram where G1(r) represents a spherical lens surface with a relatively large radius of curvature. In an IOL context, the radius of curvature of G1(r) is optimized for distance vision. G2(r) represents a spherical lens surface with a relatively small radius of curvature (relative to G1) such as optimized for near vision. One or more additional curvatures between the distance vision and near vision can also be used. In an IOL context, the smaller radius is optimized for near vision. A lens ideally can combine the optimized characteristics of both types of lenses. As mentioned, traditional methods typically involve multiple focal zone or diffractive regions each one optimized for a particular power or focal length. In non-limiting examples, a small radius corresponds to high power and near vision and can be about 17 mm, a large radius corresponds to low power and distance vision and can be about 24 mm. In another non-limiting example, distance vision means 4000 mm (pursuant to a standard eye chart distance), or infinity, or anything in between while near vision means 400 mm (pursuant to standard eye chart distance) or closer.

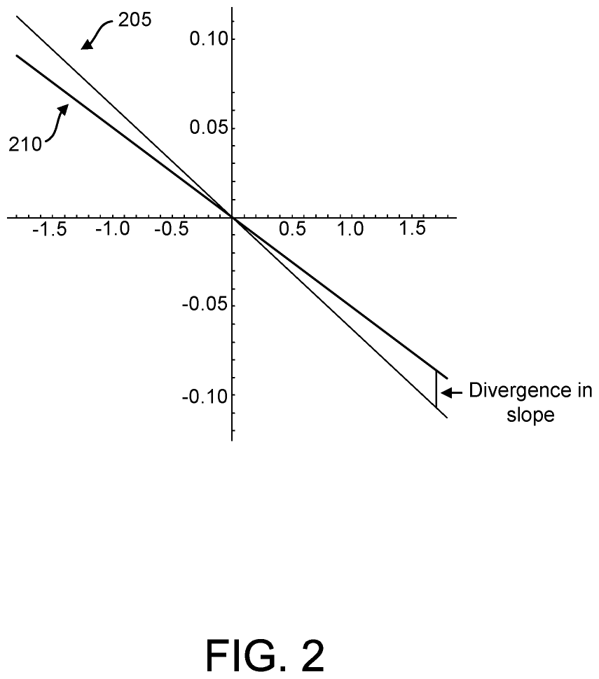

FIG. 2 shows a graph of the slope 205 of G1 and the slope 210 of G2 as a function of distance r from the optic axis of the lens. FIG. 2 illustrates that both curves have a flat (i.e., 0) slope at the optic axis and both curves have increasingly steep slopes moving further away from the optic axis. FIG. 2 also demonstrates that since G1 and G2 have different underlying radii of curvature, their slopes diverge moving further away from the optic axis. This slope divergence is proportionate to the difference in optical power between the two lenses where steeper slopes is higher power than less steep slope.

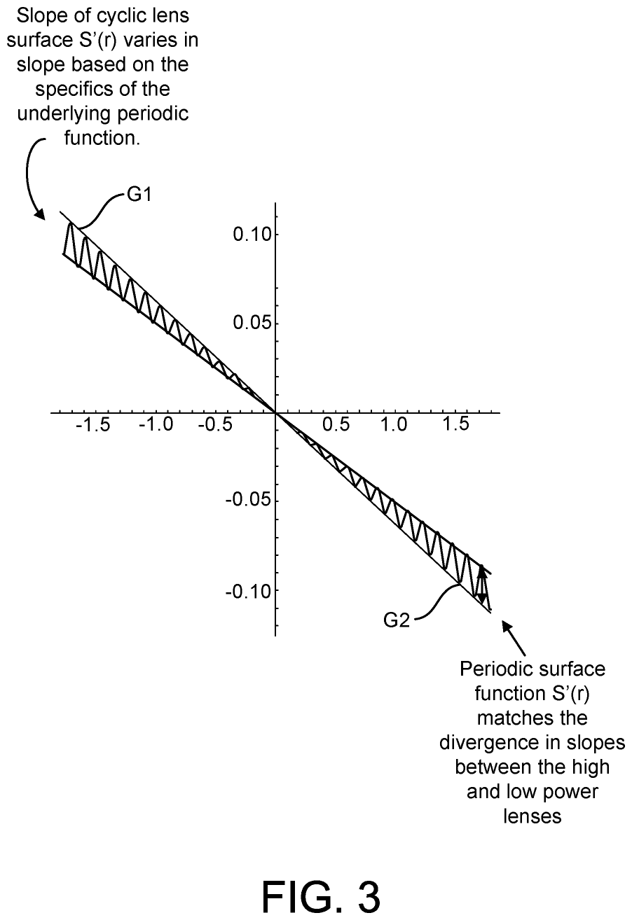

FIG. 3 shows the slope of the combined outer surface lens function S′(r) that defines the combined outer lens surface, which is the slope of the combined surface function G(r) and the function H(r). The slope varies periodically according to the amplitude and periodicity of the period function. The periodic function is chosen so that the peak and trough amplitudes vary in relation to the slopes of the high and low power lens profiles G1 and G2. The slope curves of the high power lens profile and the low power lens profile define the outer ranges of amplitude of the periodic function. In this instance, the slope maxima of the periodic profile is equal to the slope of the highest slope lens surface and the slope minima of the periodic function is equal to the slope of the lower slope lens surface. In an embodiment, an amplitude of the periodic function gets larger (i.e, increases) as radial distance from the optic axis increases. In this instance, the amplitude matches the amplitude of the divergence between the two lens surface slopes.

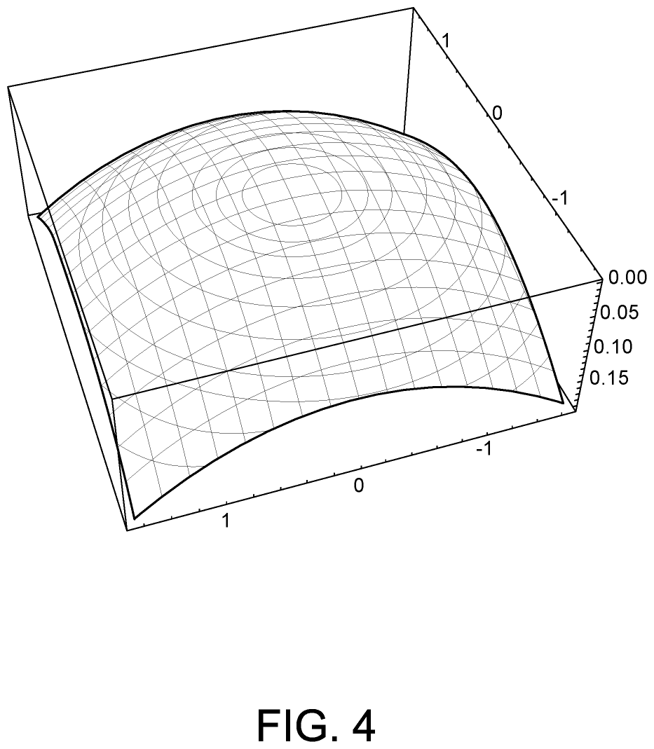

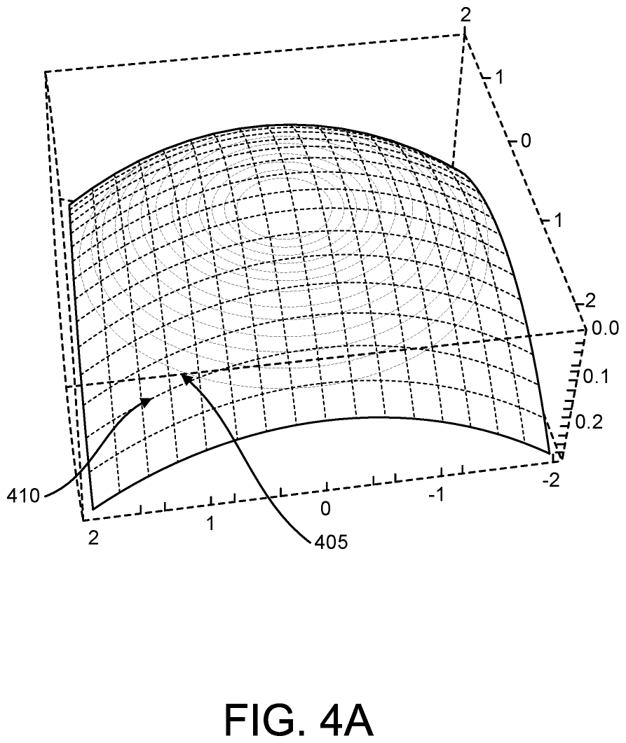

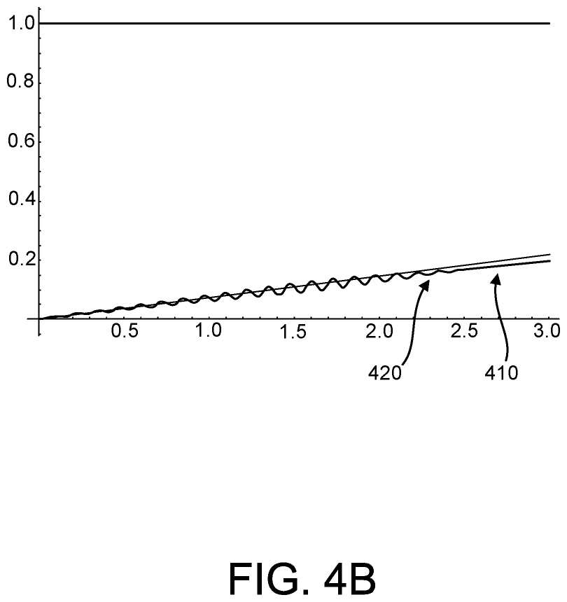

FIG. 4 shows an example representation of a combined cyclic surface as defined by the combined lens function S′(r). The underlying spherical shape is evident as are the varying, periodic surface structural features that are the result of the added function. The surface structural features enable an even distribution of high and low power across the lens surface (or any distribution of the high and low powers as defined by the combined lens function S′(r).) Further, each periodic feature contains every lens power from high to low in the system so the power distribution is much more even than in a multifocal zone lens. Note that that the combined cyclic surface has a series of periodic, annular wave shapes that are defined by the periodic function.

In an embodiment, the cyclic surface is phased out of the lens over a specific range or location of the lens. As shown FIG. 4A, a cyclic surface 405 is present on the lens. The cyclic surface 405 is present on the lens to specific radius (or location) of the lens at which point the cyclic surface decrease to 0 amplitude over a specific radial distance to form a non-cyclic surface 410. Outside this radial distance, the lens surface could be defined by a different function such as spherical surface, an aspheric surface, and/or other non-cyclic lens definition. FIG. 4B shows a graphical representation of the outer lens surface as a function of radius. The surface transitions from a combined cyclic surface at location 420 to the non-cyclic surface 410.

In another embodiment, a center region of portion of the lens is defined by a non-cyclic surface such as a spherical surface, an aspheric surface or and/or other non-cyclic lens definition. At a specific radius location of the lens, the lens transitions to a combined cyclic surface that ramps up from 0 amplitude over a specified radial distance.

In yet another embodiment, the combined cyclic surface is directly or indirectly bracketed by two non-cyclic surfaces such as a spherical surface, an aspheric surface or and/or other non-cyclic lens definition. Also, the lens can include a non-cyclic surface bracketed by two combined cyclic surfaces.

The transition between a cyclic surface and non-cyclic surface can be characterized as a transition such as a ramp up or a ramp down. A configuration of the ramp up or ramp down can be achieved by applying for example a triangle function or any function that linearly or non-linearly scales the amplitude of the cycles up or down over a specified radial distance.

Where present, the non-cyclic lens surface can be configured to achieve a specific power emphasizing a particular object distance (near vision, close vision, or intermediate vision) as desired.

According to an example method, a shape of a baseline lens surface is defined including a curvature of a distant lens base shape (referred to as the distant curvature.) A curvature of a near lens base shape (referred to as the near curvature) is also defined to achieve G(r) for both distant and near. As mentioned, additional curvatures can be defined for distances between the distant and near vision. For each of the near curvature and distant curvature (or other distances), the slope of such curvatures is calculated across the he entire lens surface or a portion thereof by obtaining the first derivative of G(r). Such slopes can be graphically represented as a function of radial distance from the lens optic axis as shown in FIG. 2, where the X-axis is the distance from the radial distance from the optic axis and the Y-axis is the slope of curvature. Note that the slopes diverge from one another moving away from the optic axis.

As mentioned, in another embodiment, a baseline lens surface shape is not predefined. A user defines one or more slope values or slope curves of the lens surface wherein the slope values/curves each relate to a desired power profile. The user may further define on or more functions to transition between the slope values/curves to achieve a combined slope curve that defines the slope of the lens surface. Mathematical integration is then used to define the lens surface. Thus, a method can include defining a first slope curve of at least a portion of the lens surface, the first slope curve relating to a lens power optimized at a first distance; defining a second slope curve of at least another portion of the lens surface, the second slope curve relating to a lens power optimized at a second distance; defining at least a third slope curve that connects the first and second slope curve; combining the first slope curve, the second slope curve and the third slope curve to obtain a combined slope curve; and integrating the combined slope curve to obtain a definition of a curvature of the lens surface

In a next step, the two (or more) slope curves or lines are connected to one another or otherwise merged using a periodic function, wherein the function can vary. In an embodiment, the function is a smooth periodic function, such as a sinusoidal function, which varies the slope of curvature smoothly between the near curvature and distant curvature. This provides an overall smooth amalgamation of power between the near slope and the distant slope. Thus, the power is smoothly encompassed between distant and near vision. In another embodiment, the function is a non-regular function such as a chirp function, in which the frequency successively increases (up-chirp) or decreases (down-chirp). In yet another embodiment, non-regular function is modified or changed in a non-linear manner such as logarithmically. In another embodiment, the function is a piecewise function.

In another embodiment, the slope of the lens can be varied arbitrarily, randomly or quasi-randomly between the slope defined by the near curvature surface and distant curvature surface (or between additional power profiles), as well as between other curvatures between near and distant. A resultant arbitrary function can be tuned to enable a specifically desired distribution of power across the lens. For example the function can be tuned to bias vision toward distant vision while retaining overall very good depth of focus. In this way the lens can be tuned in a bespoke manner allowing, any desired distribution of lens power. For example, the lens can be tuned to have one or more regions biased toward distance vision with other region(s) biased toward near vision with the regions positioned at desired locations along the lens based on the needs or desires of a lens designer.

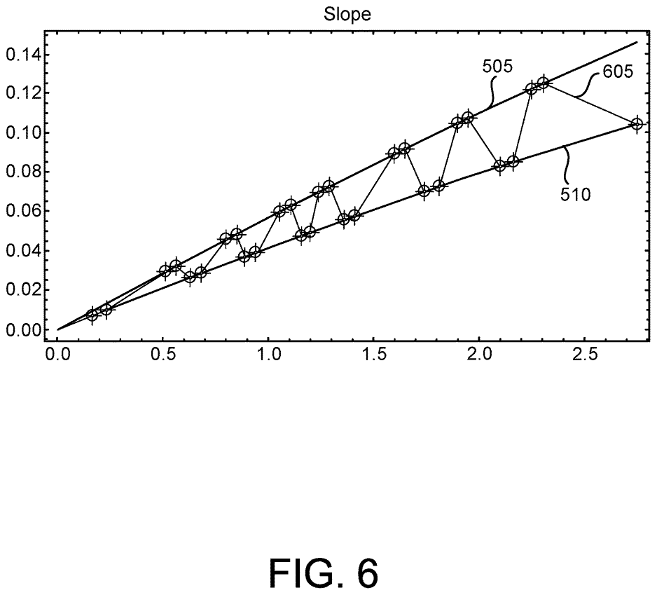

Once the appropriate function is constructed which varies the slope between distant and near vision (and possibly other visons) to achieve the desired distribution of lens power across the lens, integration is used to specify or define a shape (such as curvature) the lens surface. The function representing the combined slope as described, which could be a piecewise function comprised of line segments as shown in FIG. 6, is integrated using any appropriate integration technique. This can be accomplished symbolically, numerically, or using any other method of functional integration. The result of the integration of the slope function defines the surface of the lens, which can be at least a portion of the anterior surface, the posterior surface or a combination thereof.

It should be appreciated that the slope function can vary and can be for example a piecewise function (that is a function made up of multiple little pieces of functions). The pieces in this case can be segments (e.g., the lines 605 in FIG. 6, described below). The slope function can be any function that bounces back and forth between the distant and near slope curves. Furthermore, additional slope curves beyond near and distant slope curves can be used including any quantity of slope curves beyond near and distant curves including midway curves or any curves between near and distant slope curves.

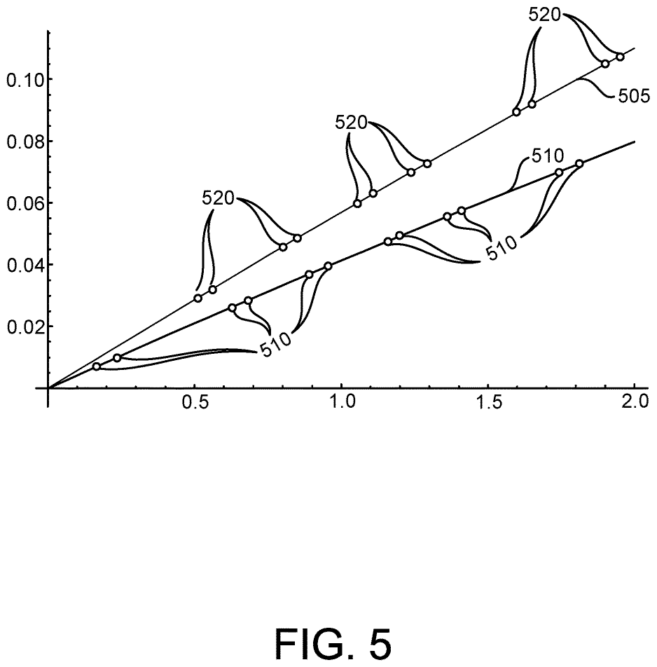

FIG. 5 shows a graphical representation of a first line 505 (or slope curve) of the slope of curvature of far vision baseline outer shape and a second line 510 (or slope curve) of the slope of curvature of far vision baseline outer shape as function of radial distance of the lens from the optic axis. In an embodiment, a user can selectively pick, choose or otherwise identify one or more transition points 520 that each represent a transition between the first line 505 to corresponding transition points 525 on the second line 510 wherein the slopes transition based on the selected periodic function. As mentioned, the period function can vary and can be regular, irregular, or arbitrary. Thus, a distance between the transition points along the first line 505 or the second line 510 can be selectively varied by a designer and can be regular, irregular, arbitrary, and/or selected by a user. In an embodiment, a user can determine the location of the transition points 520 and 525 (such as in real time) to provide the lens with a desired power profile moving radially outward from the optic axis of the lens. A user can then achieve a bespoke lens design. That is, it allows a user to achieve fine tuning of power distribution and further allows the user to achieve fine control over maximum and minim curvature of the lens surface. Accordingly, the lens power may be more consistent with varying pupil size. If the function is that defines the combines surface is regular, diffractive effects may be present due to constant width features on the lens surface. A user can overcome such diffractive effects by provide quiz random period widths to the function.

FIG. 6 schematically shows the slope curves 605 of the period function that transition between the various transition points 520 and 525 along the slope lines 505 and 510. As mentioned, a user can selectively pick the transition points 520 and 525 to achieve a bespoke power distribution for the lens. The slope curves can be random, regular, periodic or piecewise functions.

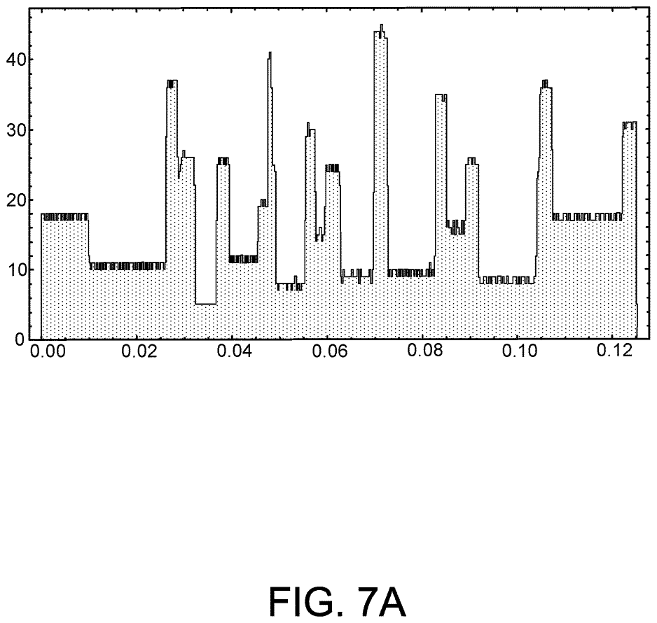

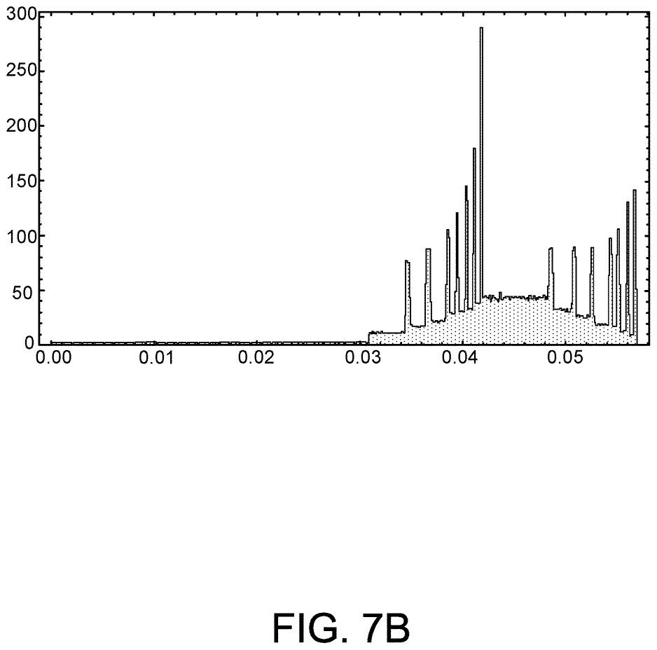

FIG. 7A shows a histogram of value of slopes of curvature across the entire lens with the horizontal axis being slope and the vertical axis being quantity of occurrence. A user can tailor an amount or quantity of a desired slope value across the lens. As an example, a vertical bar of this histogram in a non-limiting example is interpreted as “slope of 0.072 occurs 43 times across the entire lens”. This gives a metric for how much of the various powers (high low and mid for example) are present in the entire lens. In an embodiment, the user can start a lens design using the histogram such as by selectively tailoring the histogram to achieve a desired lens power distribution. The resultant histogram is then defined or otherwise represented by a slope curve that represents the slope of curvature of the lens surface based on the power profile defined by the histogram. The slope curve is integrated to achieve a definition of the lens surface. FIG. 7B shows another histogram that represents curvature of the outer surface of the lens based on the setup on FIG. 6.

In an embodiment, the representations shown in FIGS. 5, 6 and 7 can are presented in a user interface on a display such as a computer display. A user may adjust the transition points 520 and 525 to selectively achieve a desired power distribution for the lens. For example, a computer may provide a user interface that enable a user to select one or more transition points 520/525 (such as using a mouse cursor) and then slide the transition points along the curves 505/510 to selectively define the transition points. Or the user may first define one or more transition points that are not connected and then thereafter provide a function that connects or transitions the transition points to one another. In an embodiment, the user interface updates in real time as a user selectively adjusts the transition points such that that the corresponding histogram representations shown in FIGS. 7A and 7B are also updated in real time on the computer display. In this manner, the user can view a resulting histogram based on the selective adjustments of the transition points 520 and 525.

### Example Lens IOL

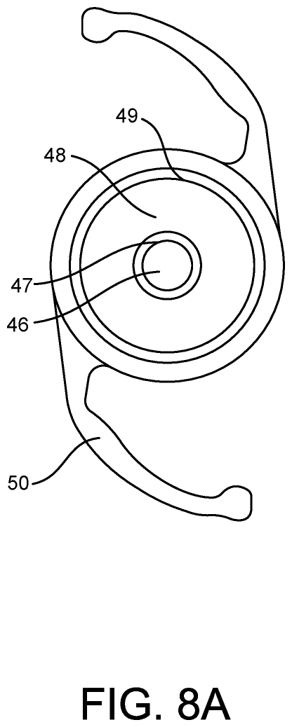

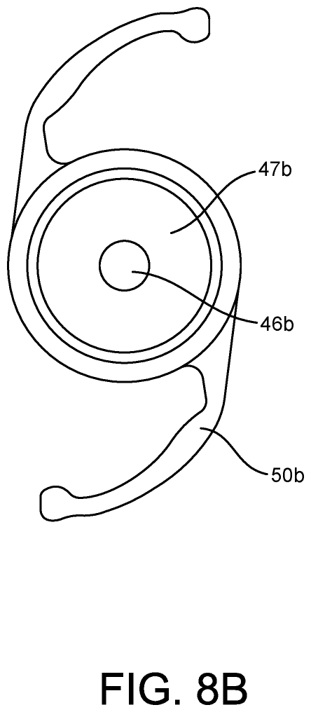

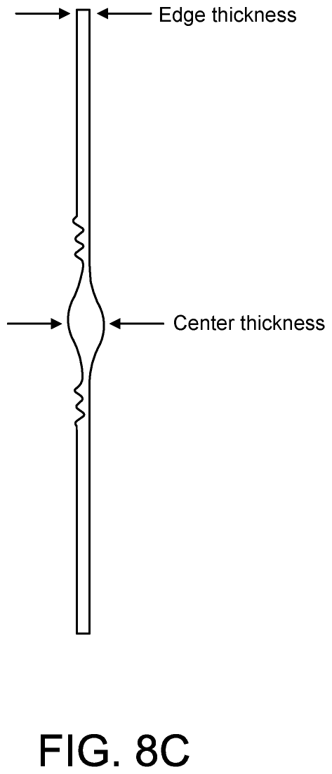

As mentioned, the lens utilizing the combined surface can vary in configuration. FIGS. 8A-8C illustrate a non-limiting example of a lens comprising an IOL. FIG. 8A shows a front view of the IOL wherein the front view may be an anterior view. FIG. 8B shows a back view of the IOL wherein the back view may be a posterior view. FIG. 8C shows a side view of the IOL. The IOL includes a central optical zone 46 (with back side 46b) that provides correction of defocus, astigmatism, and any other correction required of the lens such as spherical aberration. In a non-limiting example, for an IOL using a virtual aperture, the central optical zone diameter is smaller than that of a traditional IOL. This leads to a smaller central thickness which in turn makes the IOL easier to implant and allows a smaller corneal incision during surgery, such as an incision on the order of 2.2 mm. The central optical zone can achieve variable transmissivity of light.

The IOL optionally includes a virtual aperture 48 that is positioned further peripherally outward relative to the center location of the central optical zone 46. The virtual aperture is not really an aperture that blocks rays, but the optical effect is nearly the same on central vision. A bundle of rays incident on the virtual aperture propagate through the virtual aperture and through refraction, diffraction, scattering, reflection, and/or diffusion yield rays which are widely spread out so there is very little contribution to stray light (blurring light) at any one spot on the observation plane. The virtual aperture can be achieved via a surface modification, subsurface modification, or structure added to or positioned relative to the IOL, such as a mask structure. For example, the mask structure can be a ring-shaped structure or any ring-shaped mask that occludes at least a portion of light from passing through the IOL.

With reference still to FIG. 8A, moving peripherally outward from the virtual aperture 48, at least one IOL haptic 50 (with back side 50b) is located on the IOL. The haptic 50 can be formed of one or more arms that extend peripherally outward to define a peripheral most edge of the IOL. In an example, the optical zone has a diameter of 1.5 mm. The haptic 50 may define an outermost peripheral region of the IOL. A first plurality of light rays incident on an anterior optical surface of the optical zone can pass through the optical zone to form an image on a retina when the IOL is positioned in an eye, while a second plurality of light rays incident on an anterior virtual aperture surface are dispersed widely downstream from the IOL towards and across the retina, such that the image comprises an extended depth-of-field and further wherein the virtual aperture reduces monochromatic and chromatic aberrations in the image. The optical zone can comprise at least one of bifocal optics, trifocal optics and multifocal optics.

The virtual aperture is optionally connected to the optical zone 46 by a first transition region 47, which is located at a peripheral edge of the optical zone 46 such that the virtual aperture is a first periphery region that surrounds or partially surrounds the optical zone. The haptic can optionally comprise a second periphery region for positioning the intraocular lens within an eye. The first transition region is located peripherally outward of the optical zone 46. An optional second transition region 49 connects the haptic 50 to the virtual aperture 48. The first transition region 47 and the second transition region 49 are configured to facilitate zero- and first-order continuity of an outer surface of the IOL on either side of the respective transition region. A common way to implement these transition regions is a polynomial function such as a cubic Bezier function. Transition methods such as these are known to those skilled in the art. On the back side of the IOL is a central optic zone 46b, a haptic 50b, and a transition 47b between them. FIGS. 8A-8C are not necessarily to scale, and the haptic shape is for illustration purposes only. Other haptic shapes and sizes known to those skilled in the art would be suitable as well. The first and second transition regions are not necessarily present per se in the IOL.

The IOL has an anterior surface and a posterior surface and the components of the IOL including the optical zone 46, the first transition region 47, the second transition region 49, the virtual aperture 48, the haptic 50 can each have a respective anterior surface and posterior surface. The optical zone 46 has an anterior optical surface that can include at least one multifocal zone and/or a toric region. At least a portion or region of the anterior surface and/or the posterior surface, such as in the region of the virtual aperture or other portion of the IOL, can have a surface contour or shape that achieves a desired or predetermined effect for light passing therethrough. In nonlimiting examples, the surface contour of the anterior surface and/or the posterior surface includes a region with a ripple-type contour such as a wave shape or an undulating shape that forms a series of raised and lowered surfaces. The surface contours can achieve various effects with respect to light passing through the IOL. For example, the surface contour can achieve a wide or wider spread of stray light depending upon the type of surface contour used. The surface contour can be used to achieve a spread of stray light which is guided away from a focal point of the retina.

### Example Computing System

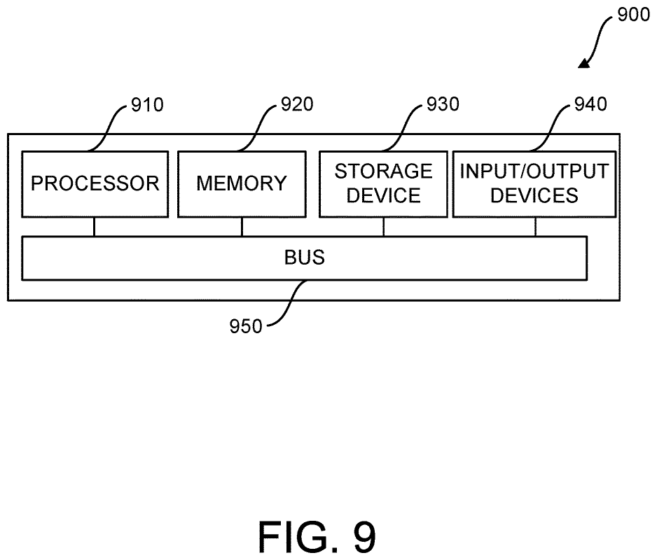

FIG. 9 depicts a block diagram illustrating an example of a computing system 900 consistent with implementations of the current subject matter. The computing system 900 may implement processes and methods described herein. The system can include or be coupled to a lens manufacturing system, an imaging system, a biothermal system, an interactive user interface, and/or an inputs program to receive and manipulate physical, biometric, biomechanical, material and mechanical information and data.

As shown in FIG. 9, the computing system 900 can include a processor 910, a memory 920, a storage device 930, and input/output device 940. The processor 910, the memory 920, the storage device 930, and the input/output device 940 can be interconnected via a system bus 950. The processor 910 is capable of processing instructions for execution within the computing system 900. Such executed instructions can implement one or more components of, for example, VESA, 3D-ID AI, and/or MP Tool. In some implementations of the current subject matter, the processor 910 can be a single-threaded processor. Alternately, the processor 910 can be a multi-threaded processor. The processor 910 is capable of processing instructions stored in the memory 920 and/or on the storage device 930 to display graphical information for a user interface provided via the input/output device 940.

The memory 920 is a computer readable medium such as volatile or non-volatile that stores information within the computing system 900. The memory 920 can store data structures representing configuration object databases, for example. The storage device 930 is capable of providing persistent storage for the computing system 900. The storage device 930 can be a floppy disk device, a digital cloud, a hard disk device, an optical disk device, or a tape device, or other suitable persistent storage means. The input/output device 940 provides input/output operations for the computing system 900. In some implementations of the current subject matter, the input/output device 940 includes a keyboard and/or pointing device. In various implementations, the input/output device 940 includes a display unit for displaying graphical user interfaces.

According to some implementations of the current subject matter, the input/output device 940 can provide input/output operations for a network device. For example, the input/output device 940 can include Ethernet ports or other networking ports to communicate with one or more wired and/or wireless networks, Bluetooth or digital cloud system (e.g., a local area network (LAN), a wide area network (WAN), the Internet).

In some implementations of the current subject matter, the computing system 900 can be used to execute various interactive computer software applications that can be used for organization, analysis and/or storage of data in various (e.g., tabular) format (e.g., Microsoft Excel®, and/or any other type of software). Alternatively, the computing system 900 can be used to execute any type of software application. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities, plug ins, or can be standalone computing products and/or functionalities. Upon activation within the applications, the functionalities can be used to generate the user interface provided via the input/output device 940. The user interface can be generated and presented to a user by the computing system 900 (e.g., on a computer screen monitor, etc.). The user interface can be integrated with other devices or virtual ecosystems.

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, joy sticks, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. An intraocular lens, comprising:
a lens body configured to be implanted in an eye, wherein the lens has an outer surface, the outer surface including a combined cyclic surface, wherein the combined cyclic surface has a first portion periodically defined by a base shape optimized at a first focal length, a second portion periodically defined by the base shape optimized at a second focal length, and a third portion that periodically connects the first and second portions, the third portion defined by a periodic function that varies in amplitude and frequency.

2. The lens of claim 1, wherein the outer surface includes a posterior surface and an anterior surface.

3. The lens of claim 1, wherein the cyclic surface is on the posterior surface.

4. The lens of claim 2, combined cyclic surface is on the anterior surface.

5. The lens of claim 1, wherein the combined cyclic surface is on just a portion of the outer surface.

6. The lens of claim 5, wherein the outer surface also includes a center region that is a non-cyclic surface such as a spherical surface or an aspheric surface.

7. The lens of claim 5, wherein the outer surface includes the combined cyclic surface bracketed by two non-cyclic surfaces such as a spherical surface or an aspheric surface.

8. The lens of claim 6, wherein the combined cyclic surface transitions to the non-cyclic surface pursuant to ramp up or a ramp down.

9. The lens of claim 1, wherein the periodic function is a sine or cosine function.

10. The lens of claim 1, wherein the periodic function is an arbitrarily large number of sine and cosine terms.

11. The lens of claim 1, wherein the periodic function is a chirp function.

12. The lens of claim 1, wherein the combined cyclic surface periodically transitions between the base shape optimized at a first focal length and the base shape optimized at a second focal length at a plurality of transition points.

13. The lens of claim 12, wherein a user can select the transition points.

14. The lens of claim 12, wherein a distance between the transition points is regular.

15. The lens of claim 12, wherein a distance between the transition points is arbitrary.

16. A lens, wherein the lens has at least one surface defined by a combination of a baseline lens shape that is combined with a periodic function modulated in amplitude and period.

17. The lens of claim 16, wherein the lens comprises an intraocular lens.

18. The lens of claim 16, wherein the at least one surface is a posterior surface.

19. The lens of claim 16, wherein the at least one surface is an anterior surface.

20. The lens of claim 16, wherein the lens comprises an endoscope.

21. The lens of claim 16, wherein the periodic function is a sine or cosine function.

22. The lens of claim 16, wherein the periodic function is an arbitrarily large number of sine and cosine terms.

23. A method of defining a lens surface, comprising:
defining a first slope curve of at least a portion of the lens surface, the first slope curve relating to a lens power optimized at a first distance;
defining a second slope curve of at least another portion of the lens surface, the second slope curve relating to a lens power optimized at a second distance;
defining at least a third slope curve that connects the first and second slope curve;
combining the first slope curve, the second slope curve and the third slope curve to obtain a combined slope curve;
integrating the combined slope curve to obtain a definition of a curvature of the lens surface.

24. The method of claim 23, wherein the first slope curve relates to a single point of the lens surface.

25. The method of claim 23, wherein the third slope curve is a periodic function.

26. The method of claim 23, wherein the third slope curve is a piecewise function.

27. The method of claim 23, wherein the third slope curve is an arbitrary function.

28. The method of claim 23, wherein the third slope curve is a piecewise function.

29. The method of claim 23, wherein the first slope relates to a region of the lens surface.

30. The method of claim 22, further comprising defining additional slope curves beyond the first slope curve, and second slope curve.
